(19) United States
(12) Patent Application Publication
(10) Pub. No.: US 2013/0231618 A1
(43) Pub. Date: Sep. 5, 2013

(54) METHOD OF TREATING SKIN

(71) Applicant: THE GILLETTE COMPANY, Boston, MA (US)

(72) Inventors: Pamela Joan ZUPKOSKY, Westford, MA (US); Helen Rochelle KEMP, Glendale, OH (US); Kristina Emma Inge VANOOSTHUYZE, Reading (GB)

(73) Assignee: THE GILLETTE COMPANY, Boston, MA (US)

(21) Appl. No.: 13/780,009

(22) Filed: Feb. 28, 2013

Related U.S. Application Data
(60) Provisional application No. 61/606,308, filed on Mar. 2, 2012.

(51) Int. Cl. A61M 35/00

(52) U.S. Cl. CPC A61M 35/00
USPC 604/290

(57) ABSTRACT

A method of treating post-shave irritation via an advanced shave regimen.

### BACKGROUND OF THE INVENTION

Various types of post shave skin irritation exist. It is believed that these irritations are particularly common with lower quality razors but some also believe that regular shaving or closer shave technologies can cause various forms of post shave irritation.

Pseudofolliculitis barbae (PFB) is a common inflammatory skin disease, particularly among African American men. This condition has been reported to occur when an ingrown facial hair triggers a foreign body inflammatory response and is characterized by the development of lesions, papules and pustules. Lesion formation and skin irritation associated with PFB can lead to men decreasing their shave frequency to 2-3 times a week. However, a low shave frequency can in turn increase the likelihood of lesion formation as the hairs are given a chance to grow long enough to continually re-enter the skin. Daily shaving minimizes hair length and can thus help reducing the opportunity of lesion formation via ingrown hairs. However, daily shaving and/or a close shave (such as via a multi-blade wet shave razor compared to a dry shave power razor) is perceived by some who have this condition as being uncomfortable. Moreover, it has been reported that many men believe that daily shaving with a multi-blade razor can exacerbate ingrown facial hair, ultimately leading to PFB.

There have been many attempts to treat or minimize the occurrence of PFB and related skin conditions by using different shaving tools as well as skin care compositions. See, e.g., U.S. Pat. Nos. 7,404,949; 5,853,709 4,944,939; 4,775,530; and 4,228,163. Additional reports describing PFB and other related skin conditions have been described in KOSMET and MEDLINE reports, such as: Zupkosky, Pamela et al., International Journal of Cosmetic Science, 2009, vol. 31, pg. 312; Garcia-Zuazaga Jorge, Military Medicine, July 2003, Vol. 168, No. 7, pg. 561-4; and Crutchfield C E 3^{rd}, Cutis; Cutaneous Medicine for the Practitioner, June 1998, Vol. 61, No. 6, pg. 351-6.

These and other post shave irritations have often been linked by many as a result of a close shave and/or regular shaving. As such, many men (and women) avoid wet shaving or decrease the frequency of shaving as they fear undesirable post shave irritation symptoms. Despite earlier attempts to address these types of skin conditions, there remains a need to better understand the relationship of shaving technologies which can provide close shaves and shave frequency with post shave irritation to allow consumers to get and maintain the clean close shave appearance they desire while avoiding one or more types of skin irritation.

### SUMMARY OF THE INVENTION

One aspect of the present invention provides for a method of treating post shave irritation, said method comprising: applying a pre-treatment composition to a portion of skin to form prepared skin; applying a first composition to said prepared skin to form treated skin; shaving said treated skin with a razor blade comprising at least three blades to form shaved skin; and applying a second composition onto said shaved skin, wherein said steps a) though c) are performed on a daily basis for at least 3 consecutive days.

Another aspect of the present invention provides for a method of treating post shave irritation, said method comprising: identifying a subject with mild to moderate post-shave itching; applying a pre-treatment composition to a portion of skin to form prepared skin; applying a first composition to said prepared skin to form treated skin; shaving said treated skin with a razor blade comprising at least five blades to form shaved skin; and applying a second composition onto said shaved skin, wherein said steps a) though c) are performed on a daily basis for at least 3 consecutive days.

Also within the scope of the present invention would any of the methods of treatment described herein, further comprising an additional step of determining a subject who has mild or moderate symptoms of one or more post shave irritations, such as post shave itching, PFB and so forth, and prescribing such a method of treatment for said subject to provide a high quality shave while avoiding undesirable post shave irritations which have been assumed by many as an unavoidable result of shaving.

### DETAILED DESCRIPTION OF THE INVENTION

1. Methods of Treatement

The present invention relates to a method of treating post shave irritation, said method comprising: applying a pre-treatment composition to a portion of skin to form prepared skin; applying a first composition to said prepared skin to form treated skin; shaving said treated skin with a razor blade comprising at least three blades to form shaved skin; and applying a second composition onto said shaved skin, wherein said steps are performed on a daily basis for at least 3 consecutive days. In one embodiment, one or more, possibly all three of the said pre-treatment composition, said first composition and said second composition, which are used across each of said consecutive days is the same—meaning for example, the user uses the same pre-treatment across all of said consecutive days, or the same first composition, or the same second composition for said consecutive days.

On a daily basis, as referred to herein, means the first instance of shaving occurs within the first day and the second instance should occur within the second day. It is possible that a user would shave at 12:01 AM on a day 1, and not again until 11:59 PM, the next day. As such, “on a daily basis can mean within a 48 hour period, preferably within a 36 hour period, preferably within a 30 hour period, preferably within a 24 hour period.

In another embodiment, the invention relates to a method of treating post shave irritation, said method comprising: identifying a subject with mild to moderate post-shave itching; applying a pre-treatment composition to a portion of skin to form prepared skin; applying a first composition to said prepared skin to form treated skin; shaving said treated skin with a razor blade comprising at least five blades to form shaved skin; and applying a second composition onto said shaved skin, wherein said treatments performed on a daily basis for at least 3 consecutive days.

In one embodiment, the method of treatment is repeated for more than 3 consecutive days, preferably at least 4 consecutive days, preferably at least 5 consecutive days, preferably at least 7 consecutive days, up to 2 weeks, up to 4 weeks, up to 8 weeks, or even up to 12 weeks. It is preferable for the user to change cartridges on a regular basis to avoid shaving with dull blades. It is believed that dull blades can create other shave related problems given new sharp blades can cut through hairs more efficiently, reducing tug and pull on the hair fibers. In one embodiment, the user or subject changes razors on a regular basis, such as every 5, 10 or even 15 shaves, before they begin to feel shave efficiency being impacted by wear on the cartridge. Without intending to be bound by theory it is now believed that where a subject follows such a shaving regimen, the occurrence of shave irritations is not exacerbated. Surprisingly, regular shaving with a comprehensive regimen of shave care products and a high performance wet shave razor, such as those having advanced blade technologies (i.e. sharpness, coatings, blade geometry, and so forth) has been observed not to adversely affect occurrence and/or severity of one or more post shave irritations.

Importantly, this allows a population of shavers who had been avoiding regular shaving with wet shave razors to confidently shave with wet shave multi-blade safety razors without fear of irritations previously assumed to be caused by such practices. It is believed that high performance razors such as 3 or 5 blade razors with advanced progressive glade geometry, such as present in Gillette Fusion® and Fusion ProGlide® and a regimen of shave products and protocols can be used together to provide one or more of these benefits.

Forms of post shave irritation include topical skin irritations including but not limited to dry skin, nicks and cuts, in grown hairs, white heads, post shave itching, redness, sensitivity, shave bumps, pseudofolliculitis barbae, and so forth.

In one embodiment of the present invention, any pre-treatment and/or first composition is applied to said portion of skin for a contact time of at least about 5 seconds, up to 300 seconds, preferably from about 10 seconds to about 120 seconds, preferably from about 30 seconds to about 60 seconds. The contact time can be cumulative between each product or relate to one or both durations of contact. In one embodiment, the pre-treatment composition can be washed off or otherwise at least partially removed prior to application of the first composition, such as where the pre-treatment is a facial or body wash or scrub. Alternatively, such as where the pre-treatment composition is a shaving oil, the oil need not be removed and the first composition can be applied over and minced in with any pre-treatment composition on the skin. Without intending to be bound by theory, it is believed that having a sufficiently long contact time allows for hairs to become hydrated and thereby soften allowing for an easier cut when contacted with the blades of the razor.

In one embodiment, the pre-treatment composition comprises at least one of a facial wash, a pre-shave scrub, and a pre-shave oil. In another embodiment, said step of applying said pre-treatment composition also includes a step of rinsing or washing off said pre-treatment composition to form said prepared skin. The step of rinsing or washing off said pre-treatment composition can include use of warm or luke warm water.

In another embodiment, the method of treatment further comprises a step of identifying a subject susceptible at least one of nicks and cuts, post shave itching, and pseudofolliculitis barbae, and wherein said portion of skin is from said subject. Without intending to be bound by theory, it is believed that the present methods of treatment can be particularly useful for such subjects as they may be avoiding regular shaving with multi-blade wet shaving razors under a belief that such avoidance helps minimize skin irritations. This is believed to be incorrect as shaving with the right protocol and regimen of products can allow a user to have a regular clean shave with less fear of undesirable post shave irritations.

Those of skill in the art will understand that the method of treatment can be conducted on any body part where shaving may be desired. Such areas include facial hair, beard hair, neck hair, scalp, underarm, legs, arms, chest, back, shoulders, forehead, and bikini area. In one embodiment, the body part or of skin is from a user/subject susceptible to mild or moderate pseudofolliculitis barbae or post shave itching. It is believed that such a method of treatment can be particularly useful for such body parts and such subjects.

2. First Composition

The first composition can be many different types of compositions which can be used in a shave preparation context. Non-limiting examples include shave foams, post foaming shave gels, non-aerosol shave gels, personal cleansers, shave oils, and even liquid or bar soaps. Non-limiting examples of compositions suitable for use as a first composition include pre-shaving moisturizers and balms as well as skin and facial cleansers; shaving oils, foams and gels; and post-shave moisturizers and lotions. Many of these compositions focus on wetting the skin and hydrating the hairs prior to shaving. It is believe that wetting or hydrating the hairs decrease the cutting force required to shave the hairs and lubricating the skin to reduce irritation which may occur from contact with the razor blade. Numerous shave preparation and cosmetic compositions have been described. See e.g. U.S. Patent Publ. Nos. 2008/0069784 and 2005/0019356A1, see also U.S. Patent Publ. 2009/0197939A1, 20090117061, 20090220625, and 20080253973; and WO 10/073278; 09/093104; and 09/094238.

3. Second Composition

The second composition can be many different types of compositions which can be used in a post-hair removal or shave context. Non-limiting examples include post shave gels or moisturizers, after shaves, cooling skin balms, sunscreen compositions, and so forth. Examples of these post shave products are also described in various publications including See e.g. U.S. Patent Publ. Nos. 2008/0069784 and 2005/0019356A1, see also U.S. Patent Publ. 2009/0197939A1, 20090117061, 20090220625, and 20080253973; and WO 10/073278; 09/093104; and 09/094238.

In one embodiment, at least two, or all three, of said pre-treatment composition, said first composition, or said second composition includes at least one same active ingredient. Suitable active ingredients for such include those disclosed in

4. Razor Blade

Various types of razor blades can be used along with the regimen of the present invention. In one embodiment, the razor blade/shaving razor is a straight edge safety razor such as those commonly used in wet shaving applications. The razor blade can have at least three blades such as a four or five blades, optionally with a trimming blade provided on the opposite side of the cartridge. Non-limiting examples of such razors which are available in the market include Gillette Fusion®, Gillette Fusion ProGlide, Gillette Venus Embrace®, as well as the Venus & Olay® 2-in-1 razor. The shaving razor can have a skin engaging member comprising a shaving aid such as a lubrication strip, and/or one or more molded shaving aids, such as the Venus Breeze® line of razors and the Schick® Intuition® line of razors. See also US Patent Publ. Nos. 2005/0011073A, 2006/225285A and 2006/080837A, and U.S. Pat. No. 7,811,553. The shaving razor can also be a composition dispensing device comprising a razor blade head unit.

5. Kits

One embodiment of the present invention provides for a kit comprising a first composition, a second composition, and a razor blade. The kit can also optionally include a set of instructions for treating skin as described herein. The kit can also comprise the pre-treatment composition.

6. Subjects Susceptible to Post Shave Irritation

Without intending to be bound by theory, it is believed that many people can benefit from the methods of treatment of the present invention. In particular, it is believed that subjects who are particularly concerned with post shave irritation can benefit from such as shaving protocol or regimen of products. In one embodiment, the method further comprises a step of identifying such a subject for use of such a treatment. Various techniques for identifying such a subject are possible. These subjects can also be self identified and be advised to use such a method of treatment.

Imaging techniques and technologies can also be used to determine subjects susceptible to pseudofolliculitis barbae. Below are non-limiting examples of imaging techniques which can be used. Of course, visual observation or self identification can be used as a step to isolate a subject suitable for such treatment.

Without intending to be bound by theory, it is believed that multi-angled imaging of a surface can allow for a comprehensive assessment which can be used for subsequent analysis and diagnosis into a skin or hair condition. This analysis can then be used to identify whether a subject has or is prone to mild or moderate post shave irritation or irritations. Viewing is intended to mean the general act of observing an object. Imaging is intended to be the actual capturing of the image via any known recordable means (including but not limited to digital or film photography, x-ray, thermographic imaging, visual inspection, and so forth).

In one embodiment, the imaging can also be done via video. In such an embodiment, the stereo imaging would be stereo video capture.

a. Viewing Step

In one embodiment of the present inventions, the method of comprises a step of viewing said portion of skin comprising at least one hair, said viewing step comprising: taking a first image of a focal point of said portion of skin from a first position, taking a second image of said portion of skin from a second position. In one embodiment, the method comprises taking a third or further subsequent image. In another embodiment, multiple shots of one or more of the images can be taken, for example the step of taking a first image can include taking two or more shots of the same image. It is believed that taking multiple shots of any of the images can be preferable should one or more of the shots be undesirably blurred or obscured during the viewing steps.

The focal point, as defined herein, refers to is the point in space where parallel light rays meet after passing through the lens of a viewing position. Since light can pass through a lens in either direction, a lens has two focal points—one on each side. The focal point in this invention refers to the position on the surface or in the vicinity of the surface which the image is taken. Preferably, the image(s) are taken while the surface is in focus and not blurry. The distance in air from the lens principal plane to the focus is called the focal length. In one embodiment, the focal point is a discrete point on the surface. In another embodiment, the focal point is chosen such that it is above or below the plane of the surface. Preferably the focal point is on the surface. The focus point, can however, rest above the surface, particularly if the focal point is chosen as a position along the length of a hair. As such, in one embodiment, the focal point is from the surface to a distance of about 5 cm above said surface, or to a distance of about 3 cm above said surface, or to a distance of about 1 cm above said surface.

In one embodiment, the surface is viewed within a predetermined amount of time from having been shaved. In one embodiment the predetermined amount of time is from about 24 to about 72 hours of having been shaved, or from about 36 to about 48 hours. By using the term about, it is intended to mean that the viewing is performed within 30 minutes of said predetermined amount of time. This is useful so consistent images can be captured, particularly if the results of multiple viewings are to be compared to one another to see if the relative health of the skin and its hairs and follicles are improving, constant, or deteriorating.

In one embodiment, the shaving done prior to the viewing is performed using a razor having at least 3 blades, or at least 5 blades. Non-limiting examples of suitable multi-blade razors include disposable and system wet shave power or manual razors commercially available from Gillette®, such as under the Mach3®, Fusion®, or Fusion Proglide® product lines. In one embodiment, the shaving preparation step is also performed using a controlled shaving preparation (such as a shave foam or post foaming gel) as well as a set shaving regimen such that the skin appears to be clean shaven so no hairs are present protruding out of the portion of skin.

In one embodiment, the surface being viewed is on the neck or under the chin/jaw line. In another embodiment, the surface being viewed is on the face. In another embodiment, the surface being viewed can be any of the underarm, the arms, chest, back, legs, pubic area, scalp, and so forth. Those of skill in the art will appreciate that this method of viewing can be performed on any portion of skin, including those portions which have hairs. Further, as explained above, the skin need not be on a human as the method can also be performed with animals.

b. Angled Views

In one embodiment, the images are taken such that an angle between the first position to the focal point and the second position and the focal point is at least about 15°, or from about 15° and about 180°, or from about 30° to about 90°, or from about 45° to about 60°. Without intending to be bound by theory, it is believed that having such an angle between the first and second positions allows for sufficient separation of the positions to allow for a three dimensional view of the surface.

In one embodiment, a line drawn between the first position and the second position (and/or any subsequent positions) is not parallel to a plane tangential to the focal point. In another embodiment, a plane drawn between the first position, the focal point, and the second position is perpendicular to a plane tangential to the focal point. Where the surface is perfectly flat, a plane, tangential to the focal point, would be the plane of the surface. Where the surface is curved, such as convexly rounded, the plane, tangential to the focal point, is a plane that goes through that focal point and that it is tangent to that focal point.

A line drawn from the first position to the focal point can range from being parallel to a plane tangential to the focal point to being perfectly normal to that plane. In one embodiment, the first position is at an angle of from about 0° to about 90°, or from about 5° to about 45°, or from about 15° to about 30°, or from about 0° to about 15° from a plane tangential to said focal point. In certain instances, it may be preferred to have the first position be relatively close to the surface and then to have the second or subsequent image(s) be taken at higher angles from the plane, tangential to the focal point. This can be particularly useful if the images are taken along a same plane which is planar to the first position and the focal point.

In one embodiment, the second position or further subsequent positions is at an angle of from about 15° to about substantially perpendicular (or normal) to said tangential plane, or from about 15° to about 75°.

Without intending to be bound by theory, by viewing the surface by taking multiple images allows for a three dimensioning of the surface along with any hairs or hair follicles there contained. It has been found that this can allow for enhanced diagnosis of skin conditions such as in grown hairs, obstructed hairs, as well as other dermatological conditions. Obstructed hairs, as referred to herein, means hairs which have grown beyond the surface of the skin and are pulling the skin immediately surrounding the hair away from the skin surface. In-grown hairs, on the other hand would be those hairs which are still contained below the surface of the skin and begin to curl and create bumps on the skin.

c. Focal Length—Distance from the Focal Point

In another embodiment, the first image is taken at substantially the same distance from the focal point as the second image. This distance is often times referred to as the focal length. In another embodiment, all images are taken at substantially the same distance such that they have substantially the same focal length. Substantially the same distance, as used herein, means that the shortest of the distances is within about 75% of the longest of the distances, preferably within about 90%, more preferably within about 95%, even more preferably within about 99%.

In another embodiment, the images can be taken at varying focal lengths. Where varying focal lengths are used, the distances of the viewing positions to the focal point should be recorded such that any subsequent analysis of the images taken can be correlated by geometry.

Those of skill in the art will understand that varying the angles and focal lengths can be done while still allowing the comparative views to provide useful information regarding the surface and any hairs or aberrations thereon. Aberrations on the surface, as referred to herein, can include skin irritations such as ingrown hairs, nicks and cuts, partially obstructed hair follicles, razor burn and so forth.

In one embodiment, the step of taking an image is taken at any focal length where a sufficiently clear image can be recorded. In one embedment the focal length can be from about 0.1 cm to about 20 cm, preferably from about 1 cm to about 15 cm, more preferably from about 2 cm to about 10 cm.

d. Magnification

In one embodiment, one or more of the images are taken under a magnification greater than 1×. In one embodiment, at least one of the images is taken at a magnification of from about 2× to about 200×, preferably from about 3× to about 50×, more preferably from about 5× to about 25×. In one embodiment, all images are taken at the about same magnification, or exactly the same magnification. When using the term “about” in reference to magnification, it is mean that the degree of magnification is within an acceptable variation based on available equipment and use conditions. In one instance, “about” a degree of magnification can be within 0.1×. In another embodiment, one or more of the images can be taken at a different magnification from the other images.

Those of skill in the art will understand that varying the magnification can allow for more intricate analysis of the surface or any hairs under the surface, or protruding out of the surface. In some instances, it can be preferable to use a magnification higher than 20× so the actual hair, hair shaft, and/or follicles can be viewed in detail. As such, in one embodiment, the images are taken at a magnification of from about 20× to about 250×, or from about 50× to about 200×, or from about 75× to about 100×.

In other instances where a more topographical analysis is desired, the magnification can be below 20×. This relative lower degree of magnification can be useful to get a broader landscape view of the surface so bumps, crevices, ridges, and other aberrations in the skin surface can be viewed. This can be particularly useful in helping identify the number and severity of PFB and in grown hairs. In one embodiment, the images are taken at a magnification of from about 10× to about 20×.

e. Luminance

Those of skill in the art will understand that varying levels of luminance can be used as long as the images taken provide discernable surface contours and conditions as well as visible hairs and hair follicle positions and conditions. In one embodiment, said step of viewing said portion of skin is performed under a luminance of from about 1,000 candelas per square meter to about 500,000 candelas per square meter, preferably from about 5,000 candelas per square meter to about 250,000 candelas per square meter, more preferably from about 10,000 candelas per square meter to about 100,000 candelas per square meter.

In another embodiment, the level of luminance is substantially constant when taking the first image and when taking the second image, preferably within 1,000 candelas per square meter.

Further, imaging could also be conducted under different lighting set ups dependant on exactly what one wishes to view without deviating from the scope and intent of the present invention. For example, these varying lighting conditions include oblique lighting (i.e. from one side of the image field), imaging under blue light (could call out specific wavelengths of light), imaging under UV black light or other light wavelengths.

f. Taking the Multiple Images

In one embodiment, the step of taking said first image and said step of taking said second image are performed simultaneously. If additional images are also taken, they can also be taken simultaneously to the first image. This can be particularly useful to minimize environmental changes which can impact the surface, such as aging, lighting changes, temperature changes, movement, tightening or loosening of skin and or muscles, and so forth. As such, it may be preferable for all or at least the first few images to be taken at the same time, or within 1 minute, or within 10 seconds, or within 5 seconds, or within 1 second of one another.

g. Imaging Technologies

i. Microscopes

In one embodiment, the step of viewing said portion of skin comprises using a microscope comprising: a) housing having a proximal end and a distal end, said housing having an opening at the distal end; b) a sensor that receives light reflected from the surface of an object being viewed and converts said light to a digital image, the sensor being located at the proximal end of the housing, c) a lens that focuses light reflected from the object onto the sensor, the lens being located between the sensor and the opening; and d) at least one reflective member. In one embodiment the at least one reflective member can be located within the opening, wherein, in use, light is reflected from the object to the sensor and a portion of said reflected light is directed via the reflective member such that the surface of the object can be viewed simultaneously from two different directions. In one embodiment, the microscope can be digital. In another embodiment, the microscope can have more than one reflective members so multiple images can be captured at the same time. Non-limiting examples of suitable digital microscopes have been described in U.S. Patent No. 2011/0058030.

In another embodiment, a microscope without a reflective member can be used. In such an embodiment, the microscope can be used to take multiple images by being repositioned as needed. In another embodiment, multiple microscopes can be used. For example, if two images are taken, a different microscope can be used for each image.

Also suitable for use herein is confocal microscopy which is an optical imaging technique used to increase optical resolution and contrast of a micrograph by using point illumination and a spatial pinhole to eliminate out-of-focus light in specimens that are thicker than the focal plane. This technique enables the reconstruction of three-dimensional structures from the obtained images.

Stereo microscopy can also be used. Stereo microscopy, in contrast to confocal microscopy using incident light illumination rather than transillumination. It uses two separate optical paths with two objectives and two eyepieces to provide slightly different viewing angles to the left and right eyes. In this way it produces a three-dimensional visualization of the sample being examined.

Those of skill in the art will understand that still cameras as well as video imaging can be used to generate the various images. In another embodiment, the surface can be viewed by the human eye, preferably assisted with a viewing lens, such as a magnifying glass or jeweler's scope. In such an embodiment, the viewer can note various factors such as length of hairs, occurrence of raised skin pulps, obstructed hair follicles, in grown hairs, and so forth. The user can then input these pieces of information into a computer for subsequent analysis, or they can analyze the data manually to provide a resultant analysis of the surface having been viewed.

7. Analysis Step

As explained above, the step of viewing said surface can result in various pieces of information being captured, recorded, or otherwise determined. Those of skill in the art will understand that the type of information desired will be based in part on the type of analysis being conducted. For example, if one were to analyze the coloration of the skin surface, the light reflectance levels of the surface would be useful to know. Non-limiting examples of useful types of information are described throughout the present application and also include, number of hairs protruding from the skin surface, thickness of hairs, diameter or cross sectional shape of hairs, length of hairs protruding from the surface, any topographical changes on the skin surface such as bumps or crevices; in grown hairs, obstructed hairs which can result in raised portions of skin; clogged pores, and so forth. Severity of a skin condition can also be determined via the above imaging or viewing techniques. For example, after running such tests on a group of subjects, skin conditions can be grouped into mild, moderate, and severe. Mild could be the 1/4 having the lowest occurrence by occurrence or severity of irritation. Moderate could be 1/2 in the middle, and severe could be the remaining 1/4 having the highest or most severe condition. The ratio could also be split into thirds if that is preferable.

In one embodiment, the method further comprises a step of analyzing said portion of skin comprising: recording the number of hairs present in said portion of skin; determining the average length of said hairs; and calculating the percentage of hairs which is less than a certain percentage of the average length, for example less than about 25%, or less than about 30%, or less than 50%, or less than 75%. The step of determining the average length of said hairs could include a step of determining the length of each hair present in the images, then taking the average of said lengths. The length of hair, as described herein, would be measured from the tip of the hair to the farthest reaching portion of skin surrounding said base of the hair, along the length of the hair. The length of said hairs can be determined by a computer using an algorithm to determine the length based on the various images recorded. The algorithm can rely on basic geometry based on the positioning with which the images or taken. The lengths can also be calculated by hand if so desired. In one embodiment, a computer can calculate the length and numbers of hairs, the average length of hairs, as well as the percentage of hairs within a set length of the average length. This program can then output a single measurement index to describe the condition of said portion of skin.

Depending on the percentage of hair which are determined to be below a certain length with respect to the average length, a rating can be assigned to that portion of skin. For example, if a portion of skin on a subject's neck has 5% to 25% of hairs less than 50% of the average hair length, then that portion of skin can be considered to be rich in in-grown hairs and/or obstructed hairs. This region of the skin can be in need of specific treatment to minimize discomfort when shaved or otherwise treated.

In another embodiment, it may be useful to focus on either the number of in-grown hairs (occurrences of PFB) and/or number of obstructed hairs. This form of analysis can be done manually or via a computer algorithm and includes the steps of: determining the number of obstructed hair and/or in grown hairs present in the portion of skin having been viewed; determining the number of hairs present in said portion of skin; and calculating the percentage of obstructed hairs and/or in grown hairs. Based on this percentage, a rating of skin/hair health can be provided. In another embodiment, other skin heath conditions can be measured and analyzed, such as occurrence of nicks and cuts, pock marks, scars, discolorations, acne, and so forth.

Another suitable step of analysis can include 1) identifying a hair by the difference in color of the hair compared to surrounding skin; 2) identifying differences in hair color along the length of the hair (above and/or below the surrounding skin); and determining whether this hair is considered obstructed based on the relative amount of hair above the surrounding hair versus the average of the other hairs.

Yet another type of analysis can include: identification of localized mounding of skin around the base of a hair or hair follicle. This type of topographical analysis could be used to determine PFB, obstructed hairs, clogged pores, acne, insect bites, puss filled pores, rashes, bacterial or fungal infections, or a multitude of other skin conditions or irritations. A similar “color” analysis step comparing any portion of hair above vs. below the surrounding skin could be used. Further, color imaging or x-ray imaging can be used to determine the presence of hairs trapped under the skin. The length of the hairs as well as the curvature under the skin can be recorded and analyzed to determine the occurrence of PFB or such conditions.

In one embodiment of the present invention, the method further comprises a step of prescribing a method of treatment according to the present invention.

8. Testing of Various Regimens for Post Shave Irritation

To demonstrate the benefits of methods according to the present invention, 90 male subjects, ages 20 to 61, with mild to moderate PFB were enrolled in a 12 week, prospective, investigator-blinded, open label, randomized clinical trial. Subjects were randomized to one of three groups:

Group 1: Shave 2-3 times per week with a commercial 3-bladed razor and standard marketed gentle shave products (a mild facial wash, a shave gel, and a post shave moisturizer);

Group 2: Shave daily with a technologically advanced 5-bladed razor (Fusion ProGlide) and standard marketed gentle shave products (same shave products as Group 1); and

Group 3: Shave daily with a technologically advanced 5-bladed razor (Fusion ProGlide) and three technologically advanced shave products (Gillette ProSeries Pre-shave wash, Gillette Fusion Shave gel, and Gillette Fusion ProSeries Post-shave lotion)

Post shave skin conditions were assessed using a combination of dermatologist, self-assessment, and instrumental measures. Statistical analyses included demographics compared by Fisher's Exact Tests and Kruskal-Wallis Tests. Outcome variables were compared using Fisher's Exact Test for number of ingrown hairs and repeated measures analysis of covariance, adjusting for baseline value of the measure and week of the reading.

69 of the 90 enrollees completed all three study visits. Results were as follows:

A significant reduction in the severity of PFB was exhibited by subjects in all 3 groups.

Group 3 subjects perceived a better improvement in the severity of PFB.

Group 3 subjects were associated with a significant reduction in post-shave itching compared to Groups 1 and 2 subjects

It should be understood that every maximum numerical limitation given throughout this specification includes every lower numerical limitation, as if such lower numerical limitations were expressly written herein. Every minimum numerical limitation given throughout this specification includes every higher numerical limitation, as if such higher numerical limitations were expressly written herein. Every numerical range given throughout this specification includes every narrower numerical range that falls within such broader numerical range, as if such narrower numerical ranges were all expressly written herein.

All parts, ratios, and percentages herein, in the Specification, Examples, and Claims, are by weight and all numerical limits are used with the normal degree of accuracy afforded by the art, unless otherwise specified.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”. All measurements are performed at 25° C., unless otherwise specified.

All documents cited in the DETAILED DESCRIPTION OF THE INVENTION are, in the relevant part, incorporated herein by reference; the citation of any document is not to be construed as an admission that it is prior art with respect to the present invention. To the extent that any meaning or definition of a term or in this written document conflicts with any meaning or definition in a document incorporated by reference, the meaning or definition assigned to the term in this written document shall govern. Except as otherwise noted, the articles “a,” “an,” and “the” mean “one or more.”

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of treating post shave irritation, said method comprising:
a. applying a pre-treatment composition to a portion of skin to form prepared skin;
b. applying a first composition to said prepared skin to form treated skin;
c. shaving said treated skin with a razor blade comprising at least three blades to form shaved skin; and
d. applying a second composition onto said shaved skin;
wherein said steps a) though d) are performed on a daily basis for at least 3 consecutive days.

2. The method of claim 1, wherein said first composition is applied to said portion of skin for a contact time of at least about 5 seconds.

3. The method of claim 1, wherein said pre-treatment composition comprises at least one of a facial wash, a pre-shave scrub, and a pre-shave oil, and wherein said step of applying said pre-treatment composition also includes a step of washing off said pre-treatment composition to form said prepared skin.

4. The method of claim 1, wherein said first composition comprises at least one of a shave foam, a post foaming shave gel, a non-aerosol shave gel, a personal cleanser, and a shave oil.

5. The method of claim 1, wherein said second composition is a post shave composition such as a balm or moisturizer.

6. The method of claim 5, wherein said second composition is not washed off.

7. The method of claim 1, wherein daily basis refers to within a 48 hour period.

8. The method of claim 1, further comprising a step of identifying a subject susceptible at least one of nicks and cuts, post shave itching, and pseudofolliculitis barbae, and wherein said portion of skin is from said subject.

9. The method of claim 1, wherein said portion of skin is selected from the group consisting of facial hair, beard/neck hair, scalp, underarm, and bikini area.

10. The method of claim 1, wherein said portion of skin is from a subject susceptible to mild or moderate pseudofolliculitis barbae.

11. The method of claim 1, further comprising a step diagnosing a subject having mild to moderate post-shave irritation.

12. The method of claim 11, wherein said step of visually diagnosis is performed visually or by an optical diagnostic machine using stereo or confocal imaging technology.

13. The method of claim 1, further comprising a step of reducing the occurrence of pseudofolliculitis barbae.

14. The method of claim 1, wherein said pre-treatment composition, said first composition and said second composition, which are used across each of said consecutive days is the same.

15. A method of treating post shave irritation, said method comprising:
a. identifying a subject with mild to moderate post-shave itching;
b. applying a pre-treatment composition to a portion of skin to form prepared skin;
c. applying a first composition to said prepared skin to form treated skin;
d. shaving said treated skin with a razor blade comprising at least five blades to form shaved skin; and
e. applying a second composition onto said shaved skin,
wherein said steps a) though e) are performed on a daily basis for at least 3 consecutive days.
